(19) United States
(12) Patent Application Publication
(10) Pub. No.: US 2018/0073087 A1
(43) Pub. Date: Mar. 15, 2018

(54) QUALITATIVE AND ABSOLUTE QUANTIFICATION KIT FOR DETECTING HEPATITIS B VIRUS CCCDNA

(71) Applicant: Yong LIAO, Chongqing (CN)

(72) Inventor: Yong LIAO, Chongqing (CN)

(21) Appl. No.: 15/559,054

(22) PCT Filed: Jun. 18, 2015
(86) PCT No.: PCT/CN2015/081800

§ 371 Date: Sep. 17, 2017

(30) Foreign Application Priority Data
Feb. 3, 2015 (CN) 201510055540.4

(51) Int. Cl. C12Q 1/70; C12Q 1/68

(52) U.S. Cl. CPC C12Q 1/706; C12Q 2600/16; C12Q 1/6876; C12Q 1/6806

(57) ABSTRACT

A composition for detecting hepatitis B virus cccDNA includes an upstream primer having the DNA sequence set forth in SEQ ID NO. 1, a downstream primer having the DNA sequence set forth in SEQ ID NO. 2, and a TaqMan probe having the DNA sequence set forth in SEQ ID NO. 3. A qualitative and absolute quantification kit for detecting hepatitis C virus cccDNA includes an extraction agent for HBV DNA; an ATP-Dependent DNase; an upstream primer having DNA sequence set forth in SEQ ID NO. 1; a downstream primer having DNA sequence set forth in SEQ ID NO. 2; a TaqMan probe having DNA sequence set forth in SEQ ID NO. 3; EvaGreen fluorescent dyes; a PCR DNA polymerase; and a digital PCR DNA polymerase.

### FIELD OF THE INVENTION

The present invention relates to the field of bioengineering, and, more particularly, to a qualitative and absolute quantitation kit for the efficient detection of hepatitis B virus DNA and hepatitis B virus covalently closed circular DNA (cccDNA) and methods thereof.

### BACKGROUND OF THE INVENTION

Polymerase chain reaction (PCR), also known as in vitro enzymatic gene amplification, was invented by Mullis in 1983. It is a sensitive, specific, rapid nucleic acid analysis technology and a breakthrough in molecular biology technology. The basic principle is to mimic the natural replication process of DNA. Primers bind with complementary DNA template in accordance with the base pairing, and under the action of DNA polymerase, according to the principle of base pairing (A, T, C, G), DNA synthesis began from the primer to synthesize strands complementary to the template DNA. After degeneration, annealing, and extension, the number of DNA strands double. Traditional PCR is a semi-quantitative, qualitative method for end-point analysis of the amplified products based on agarose gel electrophoresis. Its main advantages are simple, inexpensive, and low cost. In PCR quantitative techniques, commonly used real-time quantitative PCR is based on the accumulation of fluorescence signals during the reaction, but the quantitative PCR requires a standard reference. And it is a relatively quantitative method, and the efficiency of the amplification affects the results of quantitative PCR. Digital PCR is an absolute quantitative technique for nucleic acid molecules that can be used to accurately quantify the target DNA with high sensitivity. Digital PCR allows users to directly count the number of copies of the target DNA molecule. Digital PCR is based on traditional PCR and fluorescent probes, and does not require the establishment of standard curves and does not require reference standards to achieve highly sensitive absolute quantification of nucleotides. The main principle is to separate a PCR system into 20000 oil-water droplets. After the expansion of a regular PCR, droplets with a fluorescent signal are detected as positive, and droplets without a fluorescent signal are detected as negative. The absolute accurate quantification of the target DNA molecules is realized by statistical analysis of the Poisson distribution of the positive droplets and negative droplets in the sample.

Hepatitis B virus causes serious harm to human health. Its main replication/life process in the human body is: hepatitis B virus (HBV) binds to the liver surface receptor, its cytoplasmic antiviral capsid is removed, its incomplete closure Ring DNA (rcDNA) enters into the nucleus, under the actions of host and viral DNA polymerase, with negative strand DNA as template, DNA fissure region is extended and repaired, the two ends of the positive chain are completed to form complete double-stranded cccDNA. cccDNA transcription forms 4 Virus mRNA, and mRNA translation forms a viral protein and reverse transcription of the viral protein forms a single-stranded DNA. The negative strand DNA is then used as a template, and under the action of viral DNA polymerase, positive strand DNA is synthesized of, together with the negative chain DNA to form a new rcDNA. The latter is then packaged by the virus surface protein (capsid protein) packaging to form virus particles with the ability to infect and released to the extracellular.

cccDNA is the original template of hepatitis B virus genomic RNA replication. Although its content is less, only about 5 to 50 copies in each liver cell, it plays an important role in the replication of hepatitis B virus and the establishment of infection status. Existing nucleoside analogues antiviral drugs can not effectively remove cccDNA. If cccDNA exists in a body, HBV is likely to replicate again, leading to recurrence of hepatitis B. cccDNA is the main reason for the recurrence of HBV infection after antiviral drug treatment. In order to completely eliminate the hepatitis B virus in a body, cccDNA must be completed removed from the nucleus. Thus, it is the goal of antiviral therapy. As the intracellular cccDNA content is less, the detection is relatively difficult.

There are some methods for quantitative detection of cccDNA, mostly based on the distinction that the two chains of cccDNA are complete and gap exists in rcDNA, and a quantitative PCR.

1. Nested PCR

As a result of the use of two pairs of primers and two rounds of amplification, the first pair of primers crossed the DR1 and DR2 repeats of the cyclic HBV-DNA, and the second PCR amplified the rcDNA with the primary PCR product as a template Gap internal area. The principle of primer design is also based on the different structure of cccDNA and rcDNA. Therefore, cccDNA can be amplified, and rcDNA cannot be amplified. DONG Qingming et al (Dong Qingming, Wei Hongshan, Zhuang Hui, et al. Detection of HBV cccDNA in serum by nested polymerase chain reaction (nPCR) [J] Chinese Journal of Health Laboratory Technology, 2005, 6(3): 168-170.) applied this method to detect standard HBV cccDNA control plasmid. The results showed that 5×10⁵ copies/L HBV cccDNA could be detected by this method. However, the two-step method was prone to errors during sampling and prone to pollution, and was longer and more cumbersome.

2. Chimeric Primer Two-Step Fluorescence Quantitative PCR

Shao et al. (SHAO JB, CHEN Z, NI W Q, et al. Quantitative method to detect HBV cccDNA by chimeric primer and real-time polymerase chain reaction [J], J. Virol. Methods, 2003, 112 (1-2): 45-52) disclose the design of two sequences of chimeric primers based on the gap of non-cccDNA form of HBV genome. The 12 bases of the 3′ end are complementary to the positive strand (1604-1615), and the binding site is the downstream of the DR2 gap downstream of the negative strand gap. The 5′ end sequence is consistent with the partial sequence of the human immunodeficiency virus and does not have the same origin as the HBV genome. First, the chimeric primer was used to carry out 15 cycles of single-stranded extension of the template DNA. During this reaction, cccDNA was able to extend a new strand completely because the positive chain is complete. For other forms of HBV DNA, such as rcDNA, the extension of the primer was stopped in the DR2 region because there is a gap in DR2 region. Thus, single-stranded extension products cannot be produced. After the new strands were formed, another pair of primers was designed. One primer was hybridized with fragment B of the chimeric primer, and the other was complementary to the sequence of the positive strand DR2. This ensured that only the product of the single strand extension reaction can be detected and HBV gene cannot be directly detected. This method was used to detect positive liver tissue samples, and results were 10⁵-10⁶ copies/mL. But the problem is that the first round of amplification products, if not purified, cannot be directly used as a template. Otherwise, the complexity of the composition would directly affect the efficiency of the second round of amplification. If purification was conducted, the accuracy would be affected.

3. Intrusion Check

There were two kinds of probes designed for the target DNA. One was the initial probe, and the other was the intrusion probe. At the 5th end of the initial probe, one oligonucleotide sequence is not complementary to the target DNA. A single base at the 3′ end is not complementary to the target DNA, and flap endonuclease I cleaves the oligonucleotide sequence that is not complementary to the target DNA at the 5′ end of the initial probe. This oligonucleotide binds with a probe with fluorescence resonance energy and quenching groups, resulting in a fluorescent signal. According to this principle, intrusion probes that bind to the repeat region 2 of the positive strand of the HBV in the upstream and negative strands in the downstream were designed. Because the cccDNA positive and negative strands were complete, two kinds of fluorescent signals were generated. Because the rcDNA strands contained a gap, it can only produce one fluorescent signal. According to the fluorescence signal generated by DNA, cccDNA and rcDNA can be distinguished. Although this method is more specific, the reaction system is more complex, and the reaction efficiency is difficult to control. The time required is longer. In addition to denaturation, high start treatment was at 64° C. for 240 min.

4. Light Cycler™ Real-Time Quantitative PCR Method

Singh et al. (Singh M, Dicaire A, Wakil A E, Luscombe C, Sacks S K., Quantitation of hepatitis B virus (HBV) covalently closed circular DNA (cccDNA) in the liver of HBV-infected patients by LightCycler real-time PCR, J Virol. Methods, 2004: 118, 159-167) disclosed that Light Cycler™ real-time PCR was used to detect cccDNA in liver tissue of patients with HBV infection. The primers were designed to cross the gap of rcDNA, and a dual probe system was applied. It is characterized by the use of Plasmid-Safe™ ATP-Dependent DNase to degrade rcDNA in addition to the design of specific primers and probes in order to eliminate the interference of rcDNA nonspecific amplification to the results. The method was specific and used the Light Cycler™ real-time quantitative PCR detection system. The test results were relatively stable and reliable, and the sensitivity was 10¹ copies/mg. But the cost of probe synthesis, equipment and supplies were high, and clinical applications were limited.

5. Real-Time Quantitative PCR Detection Method

He et al. (He M L, Wu J, Chen Y, Lin M C, Lau G K, Kung H F, A new and sensitive method for the quantification of HBV cccDNA by real-time PCR, Biochem. Biophys. Res. Commun. 2002; 295: 1102-1107) disclosed a real-time quantitative PCR amplification method based the different structure and biochemical characteristics of cccDNA and rcDNA. A TaqMan probe with a light-emitting group and a quenching group was designed at the downstream of the negative-strand gap, complementary to the negative strand. Under the guidance of the upstream primer, if the negative strand was complete, the Taq enzyme would reach the site to which the Taq-Man probe binded and cleaved the probe with its exonuclease activity of 5′→3′. The quenching group lost its inhibitory effect on the 5′ end of the luminescent group, producing a fluorescent signal. On the other hand, if the negative chain contained a gap, the fluorescence signal cannot be generated because the chain extension caused by the upstream primer cannot pass through the negative chain gap. In this way, cccDNA and rcDNA can be distinguished. The linear range of its quantitative detection is 1×10²-1×10⁷ copies/L. This method can be used to analyze the sequences of 150 known A-G subtypes in the HBV gene pool. The A, B, C, F and G genotypes can be detected according to the conserved region design. Therefore, this method can be used in 90% of the Asia-Pacific region for the detection of cccDNA in hepatitis B patients. It is thought that this method is a gold standard for cccDNA quantification in clinical liver puncture specimens, but the specificity of this method is particularly low.

At present, there are patents that disclose cccDNA detection method based on real-time quantitative PCR detection method. The method was designed for HBV negative strand and fluorescence quantitative PCR detection of cccDNA. Under this method, rcDNA can also be produced. The specificity is poor, and it requires a standard sample for quantitative PCR. Further, it is a relatively quantitative technology, not an absolute quantitative technology. Sensitivity is relatively low. The qualitative detection of cccDNA is a method using Southern blot, which is a classical method of molecular biology, but the technical requirements are high. The sensitivity is low, and it is not easy to popularize in clinical practice.

It can be seen that the above quantitative methods have some shortcomings, including low sensitivity in clinical applications, poor specificity, high cost, and time-consuming and inconvenient operation. Therefore, the inventor of the present invention has established a kit and method for the absolute quantitative detection of cccDNA, which can be used to rapidly, inexpensively, and qualitatively detect cccDNA with high specificity, high sensitivity and easy operation. This method uses DNase that is safe for closed loop DNA to remove non-cccDNA (Including rcDNA, ssDNA, HBV DNA, etc.); uses a rapid, simple and inexpensive PCR to qualitatively detect cccDNA; and uses a probe method and EvaGreen fluorescent dye method to conduct a digital PCR for absolute quantitative detection of cccDNA. The EvaGreen method has less inhibition on PCR amplification and produces less non-specific amplification compared with the SybrGreen method used by other patents and literatures.

### SUMMARY OF THE INVENTION

In view of the above deficiencies, the present invention provides a qualitative and absolute quantitation kit that uses DNase that is safe for closed circular DNA to remove non-cccDNA (including rcDNA, ssDNA, HBV DNA, etc.). The kit can provide not only a rapid and inexpensive qualitative detection method for the presence of cccDNA, but also a high specificity, high sensitivity and easy operation qualitative and absolute quantitative detection method for cccDNA.

In order to achieve the above objectives, the present invention provides primers and probes for detecting hepatitis B virus cccDNA. Specifically, an upstream primer has DNA sequences set forth in SEQ ID NO. 1; a downstream primer has DNA sequence set forth in SEQ ID NO. 2; and a TaqMan probe has DNA sequence set forth in SEQ ID NO. 3.

According to the above primers and probes, the present invention provides a kit for detecting hepatitis B virus cccDNA that includes:

(1) HBV DNA extraction agent: cell lysate, Tris saturated phenol (pH: 7.6), phenol:chloroform:isoamyl alcohol=25:24:1, anhydrous ethanol, 75% ethanol, TE buffer.

(2) Closed loop DNA safe DNA enzyme (Plasmid-Safe™ ATP-Dependent DNase).

(3) Primers and probes for the gaps of rcDNA negative strand.

The upstream primer′s DNA sequence is SEQ ID
NO. 1:
5′ CTTCTCATCTGCCGGACC 3′ (nt1561-1579).
The downstream primer′s DNA sequence is SEQ ID
NO: 2:
5′ CACAGCTTGGAGGCTTGA 3′ (nt1865-1883).
The TaqMan probe′s DNA sequence is SEQ ID
NO: 3:
FAM-5′ AGGCTGTAGGCATAAATTGGTCT 3′-BHQ (nt 1838-
1861).

(4) EvaGreen fluorescent dyes.

(5) General PCR DNA polymerase (2×Power Taq PCR MasterMix, Baxter).

(6) Digital PCR DNA polymerase (Droplet PCR Supermix, BIO-RAD).

Using closed loop DNA safe DNA enzyme (Plasmid-Safe™ ATP-Dependent DNase) for cccDNA qualitative detection and cccDNA digital PCR absolute quantitative detection:

1. cccDNA Common PCR Qualitative Detection:

(1) Primer design: Primers were designed for the negative strand of rcDNA.

Upstream primer:
(SEQ ID NO. 1)
5′ CTTCTCATCTGCCGGACC 3′ (nt 1561-1579)
Downstream primer:
(SEQ ID NO. 2)
5′ CACAGCTTGGAGGCTTGA 3′ (nt 1865-1883)

(2) HBV DNA xxtraction of: DNA extraction was carried out in a conventional manner using the extraction agents for HBV DNA in the above kit.

(3) Closed loop DNA safe DNA digestion (Plasmid-Safe™ ATP-Dependent DNase): The DNA extracted in step (2) was treated with Plasmid-Safe™ ATP-Dependent DNase to effectively degrade rcDNA and ssDNA that contain gaps. cccDNA was not affected. This reduces the rcDNA-induced nonspecific amplification, reduces rcDNA background content, and improves the specificity of the detection. Digestion system: 3 μg DNA, 5 μL 10×buffer, 2 μL 25 m MATP, 10 U Plasmid-Safe™ ATP-Dependent DNase, adding ddH₂O to 50 μL. Keeping at 37° C. for 30 min, and then at 70° C. for 30 min for enzyme inactivation.

(4) PCR Reaction

Reaction system: 1 μL of 10 μM upstream primer, 1 μL of 10 μM downstream primer, 2×PCR reaction DNA polymerase 10 μL, DNA1 μL, adding ddH₂O to 20 μL.

Reaction conditions: preheated at 95° C. for 3 min, 95° C. for 30 s, 58° C. for 1 min, 72° C. for 30 s, and 35 cycles at 98° C. for 10 min.

(5) Agarose Gel Electrophoresis

2. cccDNA Digital PCR Absolute Quantitative Detection Method:

(1) Primers and Probes design: based on rcDNA negative strand that contains gaps.

Upstream primer:
(SEQ ID NO. 1)
5′ CTTCTCATCTGCCGGACC 3′ (nt 1561-1579)
Downstream primer:
(SEQ ID NO. 2)
5′ CACAGCTTGGAGGCTTGA 3′ (nt 1865-1883)
TaqMan probe DNA sequence is set forth in SEQ ID
NO: 3:
FAM-5′ AGGCTGTAGGCATAAATTGGTCT 3′-BHQ (nt 1838-
1861)

(2) HBV DNA extraction of: DNA extraction was carried out in a conventional manner using the extraction agent for HBV DNA in the above kit.

(3) Closed loop DNA-safe DNA enzyme digestion: The DNA extracted in step (2) was treated with a closed-loop DNA-safe DNA enzyme (Plasmid-Safe™ ATP-Dependent DNase). rcDNA and ssDNA were effectively degraded, and cccDNA was not affected. This reduces the rcDNA-induced nonspecific amplification, reduces rcDNA background content, and improves the specificity of the detection. Digestion system: 3 μg DNA, 5 μL 10×buffer, 2 μL 25 m MATP, 10 U closed circular DNA safe DNAase (Plasmid-Safe™ ATP-Dependent DNase), adding ddH₂O to 50 μL. Keeping at 37° C. for 30 min, and at 70° C. for 30 min for enzyme inactivation.

(4) Using an EvaGreen fluorescent dye method or a probe method for digital PCR reaction.

### Advantageous of the Present Invention

1. Regular PCR was used for the qualitative detection of cccDNA, and the reaction time is about 70 min. The method is time-saving, convenient, and low cost. Traditional Southern blot method takes several days, and is time-consuming and cumbersome. The kit of the present invention makes it possible to quickly determine whether cccDNA is completely cleared after clinical treatment.

2. The sensitivity of digital PCR reaction: Digital PCR detection of HBV plasmid DNA is in the range of 10⁵-10¹ copy/μL.

3. The specificity of the reaction: After closed-loop DNA-safe DNase was digested by ECOR1, there was not PCR product. Without ECOR1 digestion, there was PCR product. This indicated that the specificity of PCR was increased after adding the closed loop DNA-safe DNase.

### BRIEF DESCRIPTION OF THE DRAWINGS

In the drawings:

FIG. 1 is a design and schematic diagram of PCR primers and probes for the detection of cccDNA.

FIG. 2 is a diagram that shows HBV replication process and the action of covalent closed-loop DNA safe DNase.

FIG. 3 is the PCR diagram of HBV plasmid DNA with or without covalent closed-loop DNA-safe DNase at different concentrations. A: the PCR results after treating HBV plasmid with EcoRI; B: the PCR results after treating HBV plasmid with EcoRI and covalent closed-loop DNA safe DNase.

FIG. 4 is the PCR diagram of cccDNA of the DNA of HepG2.215 cells with or without covalent closed-loop DNA-safe DNase.

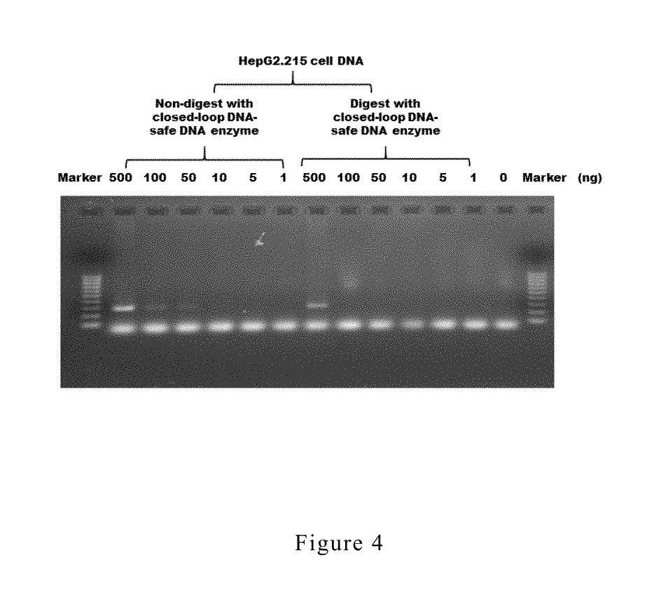

FIG. 5 shows the sensitivity results of digital PCR detection.

### DETAILED DESCRIPTION OF THE ILLUSTRATED EMBODIMENTS

A qualitative and absolute quantitation kit for the detection of cccDNA by using a closed-loop DNA safe DNA enzyme is described in detail below (any agents that are not specifically described in this kit are commercially available).

HBV DNA extraction agent: cell lysate, Tris saturated phenol (pH: 7.6), phenol:chloroform:isoamyl alcohol=25:24:1, anhydrous ethanol, 75% ethanol, TE buffer.

Plasmid-Safe™ ATP-Dependent DNase:

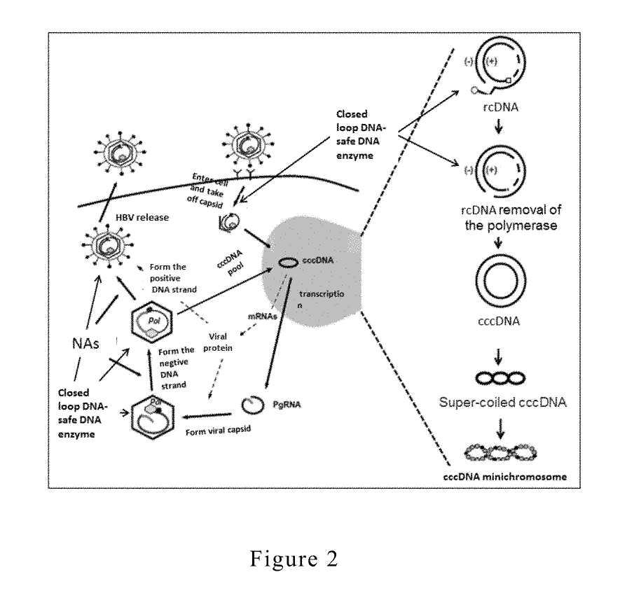

It effectively degrades rcDNA and ssDNA that contain gaps; has no effect on cccDNA; reduces rcDNA-induced nonspecific amplification; reduces rcDNA background content; and improves the specificity of the reaction. See FIG. 2.

Primers and Probes:

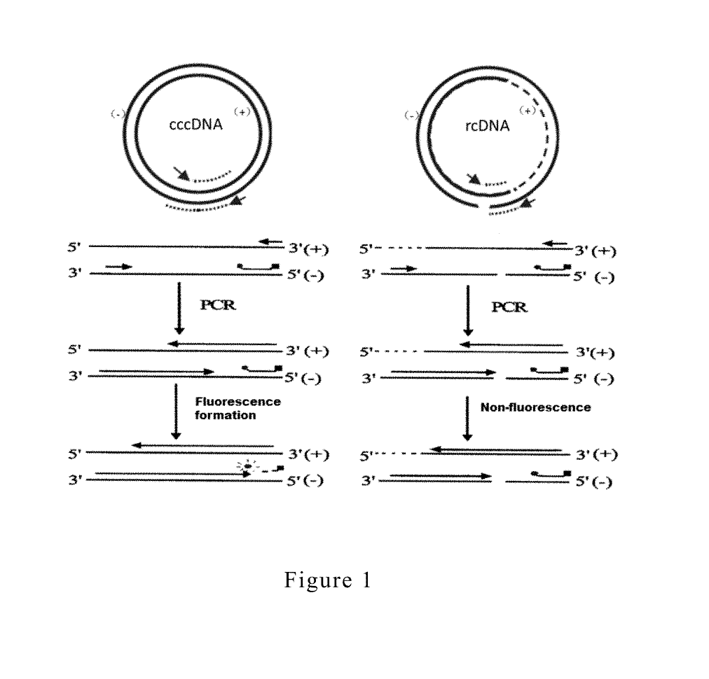

Primers and probes were synthesized. See FIG. 1. HBV cccDNA is completely closed-loop DNA, and HBV rcDNA is not completely closed-loop DNA. Primer and probes were designed based on HBV rcDNA negative strand that contains gaps. The primer sequences are as follows:

Upstream primer:
(SEQ ID NO. 1)
5′ CTTCTCATCTGCCGGACC 3′ (nt 1561-1579),
Downstream primer:
(SEQ ID NO. 2)
5′ CACAGCTTGGAGGCTTGA 3′ (nt 1865-1883),
TaqMan probe has a DNA set forth in SEQ ID
NO. 3:
FAM-5′ AGGCTGTAGGCATAAATTGGTCT 3′-BHQ (nt 1838-
1861) (FAM(6-carboxyfluorescein) and BHQ (Black
Hole Quencher) not shown in SEQ ID NO. 3);

EvaGreen fluorescent dyes;

PCR product: 332 bp.

DNA polymerase (2× Power Taq PCR MasterMix, Baxter) required for regular PCT reaction.

PCR Supermix (following BIO-RAD digital PCR operation instructions) required for digital PCR reaction.

A qualitative detection method of cccDNA and an absolute quantitative digital PCR detection method of cccDNA using closed-loop DNA safe DNA enzyme (Plasmid-Safe™ ATP-Dependent DNase):

A qualitative detection method of cccDNA using regular PCR:

1.1 HBV DNA Extraction:

(1) 150 mmol/L NaCl, 50 mmol/L Tris-HCl (pH 7.4), 10 mmol/L EDTA, 0.1% SDS Proteinase K (800 g/mL), kept at 37° C. overnight.

(2) adding same volume of Tris saturated phenol 500, mixing up and down thoroughly, and then centrifuged at 4° C., 12000 r/min for 10 min.

(3) taking upper aqueous layer 500 μL, adding same volume of phenol:chloroform:isoamyl alcohol=25:24:1 mixture 500 μL, mixing up and down thoroughly, keeping at room temperature for 19 min, and then centrifuged at 4° C., 12000 r/min for 10 min.

(4) after centrifugation, taking upper aqueous layer 450 μL, adding 4° C. pre-cooled 1/10 volume of sodium acetate 45 μL (3 mol/L, pH=5.2) and 2 times the volume of anhydrous ethanol (4° C. precooling) 1.1 mL, mixing up and down thoroughly, keeping at −20° C. for 1 h, and centrifuged at 4° C., 12000 r/min for 15 min.

(5) discarding the supernatant, adding 1 mL 70% ethanol, and centrifuged at 4° C., 12000 r/min for 10 min.

(6) drying to remove remaining ethanol (about 30 min), adding TE buffer 40 μL, and frozen for future use.

1.2 Design and Synthesis of Primers:

The design of the primer is shown in FIG. 1. Primers were synthesized and their sequences are shown below:

Upstream primer:
(SEQ ID NO. 1)
5′ CTTCTCATCTGCCGGACC 3′ (nt 1561-1579),
Downstream primer:
(SEQ ID NO. 2)
5′ CACAGCTTGGAGGCTTGA 3′ (nt 1865-1883).

1.3 cccDNA Safe DNAase (Plasmid-Safe™ ATP-Dependent DNase) Purification:

pcDNA3.1-HBV1.3 plasmid was diluted to 10⁷-10¹ copy/μL. The DNA of hepg2.215 cells was diluted to 500 ng, 100 ng, 50 ng, 10 ng, 5 ng, 1 ng. Digestion system: 3 μg DNA, 5 μL 10×buffer, 2 μL 25 m MATP, 10 U DNase, adding ddH₂O to 50 μL, keeping at 37° C. for 30 min, and at 70° C. for 30 min for enzyme inactivation.

1.4 PCR Amplification:

Reaction system: 1 μL of 10 μM upper and 1 μL of 10 μM lower primer, 10 μL 2×PCR reaction DNA polymerase, adding ddH₂O to 20 μL.

Reaction conditions: preheating at 5° C. for 3 min, at 95° C. for 30 s, at 58° C. for 1 min, at 72° C. for 30 s, and 35 cycles at 98° C. for 10 min.

1.5 Agarose Gel Electrophoresis

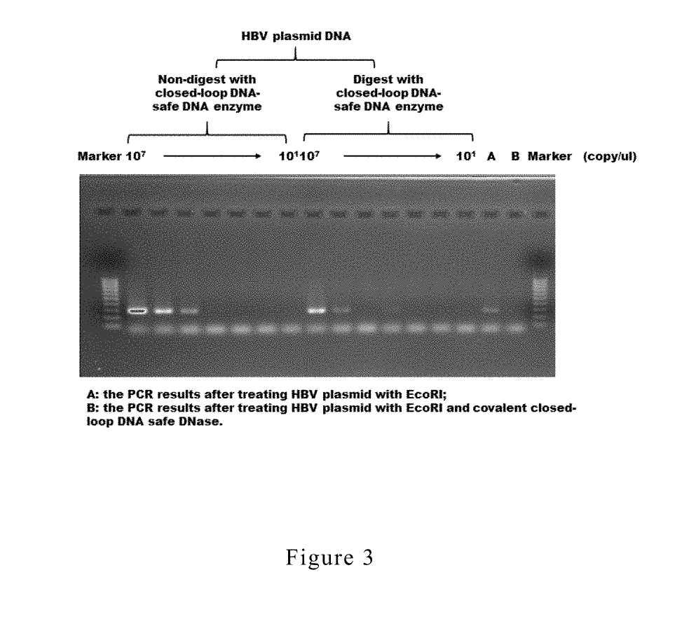

HBV plasmid DNA and HepG2.215 cells were tested according to the above steps. The results are shown in FIG. 2 and FIG. 3. The PCR results of HBV DNA at different concentrations and of hepg2.215 cells at different concentrations showed that cccDNA could be amplified by this method and the non-specific amplification was significantly reduced after digestion with Plasmid-Safe™ ATP-Dependent DNase. There was no PCR product after the digestion by EcoRI. This indicated that Plasmid-Safe™ ATP-Dependent DNase plays an important role in the specificity of cccDNA amplification in PCR amplification of cccDNA.

Absolute quantitative detection of cccDNA based on digital PCR using a closed-loop DNA safe DNA enzyme (Plasmid-Safe™ ATP-Dependent DNase)

2.1 HBV DNA Extraction:

(1) 150 mmol/L NaCl, 50 mmol/L Tris-HCl (pH 7.4), 10 mmol/L EDTA, 0.1% SDS Proteinase K (800 g/mL), kept at 37° C. overnight.

(2) adding the same volume of Tris saturated phenol 500, mixing up and down thoroughly, and then centrifuged at 4° C., 12000 r/min for 10 min.

(3) taking upper aqueous layer 500 μL, adding same volume of phenol:chloroform:isoamyl alcohol=25:24:1 mixture 500 μL, mixing up and down thoroughly, keeping at room temperature for 19 min, and then centrifuged at 4° C., 12000 r/min for 10 min.

(4) after centrifugation, taking upper aqueous layer 450 μL, adding 4° C. pre-cooled 1/10 volume of sodium acetate 45 μL (3 mol/L, pH=5.2) and 2 times the volume of anhydrous ethanol (4° C. precooling) 1.1 mL, mixing up and down thoroughly, keeping at −20° C. for 1 h, and centrifuged at 4° C., 12000 r/min for 15 min.

(5) discarding the supernatant, adding 1 mL 70% ethanol, and centrifuged at 4° C., 12000 r/min for 10 min.

(6) drying to remove remaining ethanol (about 30 min), adding TE buffer 40 μL, and frozen for future use.

2.2 Design and Synthesis of Primers:

The design of the primer is shown in FIG. 1. Primers were synthesized and their sequences are shown below:

Upstream primer:
(SEQ ID NO. 1)
5′ CTTCTCATCTGCCGGACC 3′ (nt 1561-1579),
Downstream primer:
(SEQ ID NO. 2)
5′ CACAGCTTGGAGGCTTGA 3′ (nt 1865-1883).

2.3 cccDNA Safe DNAase (Plasmid-Safe™ ATP-Dependent DNase) Purification:

pcDNA3.1-HBV1.3 plasmid was diluted to 10⁵-10¹ copy/μL. The DNA of hepg2.215 cells was diluted to 500 ng, 100 ng, 50 ng, 10 ng, 5 ng, 1 ng. Digestion system: 3 μg DNA, 5 μL 10×buffer, 2 μL 25 m MATP, 10 U DNase, adding ddH₂O to 50 μL, keeping at 37° C. for 30 min, and at 70° C. for 30 min for enzyme inactivation.

2.4 PCR Amplification:

Reaction system: 1 μL of 10 μM upper and 1 μL of 10 μM lower primer, 10 μL 2×digital Supermix, 1 μL NDA, adding ddH₂O to 20 μL.

EvaGreen dye method reaction system: 1 μL of 10 μM upper and 1 μL of 10 μM lower primer, 10 μL 2×digital Supermix, 1 μL 20×fluorescent probe, 1 μLL DNA, adding ddH₂O to 20 μL.

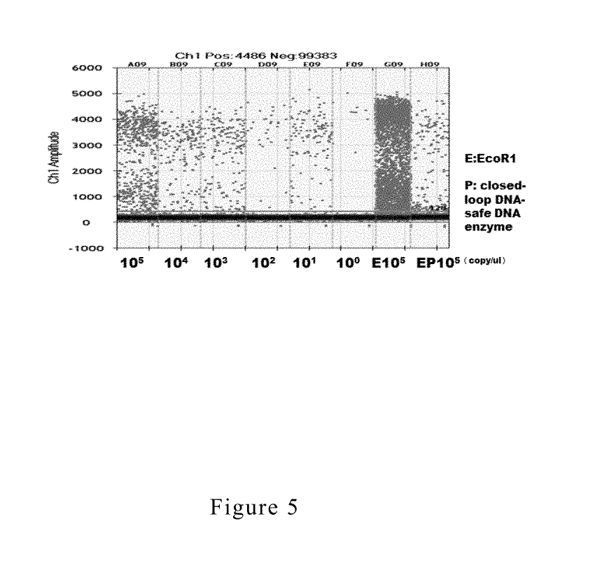

Reaction conditions: preheating at 5° C. for 3 min, at 95° C. for 30 s, at 58° C. for 1 min, at 72° C. for 30 s, and 35 cycles at 98° C. for 10 min (following BIO-RAD digital PCR instructions). Results are shown in FIG. 5. cccDNA was quantitatively quantified by digital PCR, and the lower limit of detection of cccDNA could reach a single copy. This indicated that the absolute quantification method had a high sensitivity. Each black dot in the figure represents a copy, and the numerical processing system of the specific numerical PCR reaction instrument automatically gave the number of the copies.

1. A composition for detecting hepatitis B virus cccDNA, comprising an upstream primer having the DNA sequence set forth in SEQ ID NO. 1, a downstream primer having the DNA sequence set forth in SEQ ID NO. 2, and a TaqMan probe having the DNA sequence set forth in SEQ ID NO. 3.

2. A qualitative and absolute quantification kit for detecting hepatitis B virus cccDNA comprising
an extraction agent for HBV DNA;
an ATP-Dependent DNase;
an upstream primer having DNA sequence set forth in SEQ ID NO. 1;
a downstream primer having DNA sequence set forth in SEQ ID NO. 2;
a TaqMan probe having DNA sequence set forth in SEQ ID NO. 3;
EvaGreen fluorescent dyes;
a PCR DNA polymerase; and
a digital PCR DNA polymerase.

3. The qualitative and absolute quantification kit for detecting hepatitis C virus cccDNA of claim 1, wherein the extraction agent for HBV DNA includes a cell lysate, a Tris saturated phenol, a mixture of phenol:chloroform:isoamyl alcohol=25:24:1, anhydrous ethanol, 75% ethanol, and TE buffer.


SEQUENCE LISTING (3 sequences)

SEQ ID NO: 1 - DNA, 18 bp, Artificial Sequence
  DNA Sequence of upstream primer
cttctcatctgccggacc

SEQ ID NO: 2 - DNA, 18 bp, Artificial Sequence
  DNA Sequence of downstream primer
cacagcttggaggcttga

SEQ ID NO: 3 - DNA, 23 bp, Artificial Sequence
  DNA Sequence of TaqMan primer
aggctgtaggcataaattggtct